(19) United States
(12) Patent Application Publication
(10) Pub. No.: US 2002/0160952 A1
(43) Pub. Date: Oct. 31, 2002

(54) Inhibition of protein-protein interaction

(76) Inventors: Aleksey G. Kazantsev, Boston, MA (US); David E. Housman, Newton, MA (US); Leslie M. Thompson, Irvine, CA (US); J. Lawrence Marsh, Newport Beach, CA (US)

Correspondence Address: ERIC L. PRAHL, FISH & RICHARDSON P.C., 225 Franklin Street, Boston, MA, 02110-2804, US

(21) Appl. No.: 09/933,638

(22) Filed: Aug. 20, 2001

Related U.S. Application Data
(60) Provisional application No. 60/226,502, filed on Aug. 18, 2000.

(30) Foreign Application Priority Data
Aug. 20, 2001 (US) PCT/US01/26097

(51) Int. Cl. A61K 38/00; C07K 14/00

(52) U.S. Cl. 514/12; 530/350

(57) ABSTRACT

The invention features inhibition of protein-protein interaction by therapeutic agents, which can be used to treat numerous disorders, including those associated with expanded CAG repeats.

### CLAIM OF PRIORITY

This application claims priority under 35 USC §119(e) to U.S. patent application Ser. No. 60/226,502, filed on Aug. 18, 2000, the entire contents of which are hereby incorporated by reference.

### GOVERNMENT SUPPORT

The work described herein was supported in part by a grant from the National Institutes of Health (PO1-CA42063). The United States government may, therefore, have certain rights in the invention.

### TECHNICAL FIELD

The field of the invention is inhibition of protein-protein interaction by therapeutic agents, which can be used to treat numerous disorders, including those associated with expanded CAG repeats.

### BACKGROUND

At least eight progressive, inherited neurodegenerative disorders are caused by an expansion of the naturally occurring CAG tract that codes for a polyglutamine (polyQ) repeat within the coding region of the corresponding protein. These diseases include Huntington's disease (HD), spinal and bulbar muscular atrophy (SBMA; also known as Kennedy's disease), dentatorubral-pallidoluysian atrophy, spinocerebellar ataxia type 1 (SCA1), SCA2, SCA6, SCA7 and Machado-Joseph disease (MJD/SCA3)(Reddy et al. Trends Neuroscience 22:248-255, 1999). With the exception of SCA6 (CACNLlA4)(Zhuchenko et al. Nature 15:62-69, 1997), which is characterized by a minimal repeat expansion, affected individuals show a similar range of repeat expansion above ˜35 repeats (Kakizuka et al. Trends Genet. 14:396-402, 1998).

Each disorder is inherited as an autosomal dominant (or X-linked in the case of SBMA), neurological syndrome with selective, neuronal cell death resulting in distinct, but overlapping, clinical and pathological manifestations (Ross et al. Neuron 19:1147-1150, 1997). Age of onset is normally in mid-life; however, longer repeat ranges can cause more severe presentation of the disease with an earlier age of onset. Genetic studies provide evidence that inactivation of a single allele does not result in disease (Duyao et al. Science 269:407-410, 1995; Zeitlin et al. Nature Genet. 11:155-163, 1995). In addition, mouse models for HD, SCA-1 and MJD (Reddy et al. Trends Neuroscience 22:248-255, 1999; Burright et al. Cell 82:937-948, 1995; Hodgson et al. Neuron 23:181-192, 1999; Ikeda et al. Nature Genet. 13:196-202, 1996; and Mangiarini et al. Cell 87:493-506, 1996), carrying expanded repeat transgenes in a background with two normal alleles, show phenotypes resembling the corresponding disease suggesting a true dominant effect. The appearance of neuronal intranuclear inclusions that contain huntingtin and ubiquitin, in mice transgenic for exon 1 of huntingtin, implicates protein misfolding and aggregation as potential mediators of neuronal pathogenesis (Davies et al. Cell 90:537-548, 1997). These insoluble neuronal aggregates and nuclear inclusions have been described for many of the polyQ repeat diseases, having been seen in the affected regions of brains from patients (Kakizuka et al. Trends Genet. 14:396-402, 1998; DiFiglia et al. Science 277:1990-1993, 1997; Bates et al. Brain Pathol. 8:699-714, 1998; Paulson et al. Am. J Hum. Genet. 64:339-345, 1999) and in most of the transgenic mouse models (Bates et al. Brain Pathol. 8:699-714, 1998; Paulson et al. Am. J. Hum. Genet. 64:339-345, 1999).

A role for nuclear localization of expanded polyQ repeat-containing disease proteins, independent of aggregation, has also been implicated in the initiation of disease and neurodegeneration (Klement et al. Cell 95:41-53, 1998; Saudou et al. Cell 95:55-66, 1998). In contrast, the presence of cytosolic aggregates in dystrophic neurites and neuropils in HD brain sections and in HD transgenic mice may reflect a pathogenic role for non-nuclear localization and aggregation (DiFiglia et al. Science 277:1990-1993, 1997; Gutekunst et al. J. Neurosci. 19:2522-2534, 1999; Li et al. Hum. Mol. Getter. 8:1227-1236, 1999). The aggregation phenomenon has been reproduced in vitro in a protein concentration and repeat length-dependent manner (Scherzinger et al. Proc. Natl. Acad. Sci. USA 96:4604-4609, 1999), demonstrating that aggregation is a property mediated by the expanded polyQ. The structure and behavior of polyQ repeats, both isolated and in protein contexts, have been examined in vitro; these studies argue that a structural transition associated with increased length occurs to mediate aggregation (Perutz et al. Trends Biochem. Sci. 24:58-63, 1999).

### SUMMARY

The present invention is based on the discovery of therapeutic agents that can be used to prevent protein-protein interaction (e.g., aggregation, dimerization, or other physiologically significant association). Thus, the agents can be used to treat Alzheimer's disease, disorders associated with expanded CAG repeats (such as Huntington's Disease), and disorders in which polyglutamine-containing transcription factors or coactivators are undesirably active (e.g. disorders associated with homodimerization of jun or hexamerization of p53).

The therapeutic agents contain three domains. The first and second domains bind to proteins (e.g., cytosolic or nuclear proteins) and the third domain separates the first and second domains such that the proteins bound to the first and second domains do not interact with one another as they otherwise would. In one embodiment, the first and second domains bind to proteins, such as the proteins encoded by huntingtin, that contain at least seven consecutive glutamine residues (e.g., 7, 10, 15, 20, 25, 30, 35, 36, 37, 38, 39, or 40 or more consecutive glutamine residues). When administered to a patient, the agent inhibits aggregation of proteins with abnormally expanded regions of polyglutamine.

In other embodiments, the therapeutic agents inhibit the interaction between polyglutamine-containing proteins such as transcription factors and coactivators, or between tau proteins. For example, the first and second domains of a therapeutic agent can consist of a peptide, such as VQIVYK (SEQ ID NO:1), which binds to and prevents the aggregation of tau proteins. Such an agent is useful for the treatment of Alzheimer's disease.

The details of one or more embodiments of the invention are set forth in the materials that follow. Other features, objects, and advantages of the invention will be apparent from the description, the drawings, and the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated herein by reference in their entirety.

### BRIEF DESCRIPTION OF THE DRAWINGS

FIG. 1 is a representation of the Tata-binding protein (SEQ ID NO: 12; TBP; encoded by Genbank Accession No. M55654). The alpha-helical regions H1 (LKTIALRAR; SEQ ID NO:2), H2 (EEQSRLAARKYAR; SEQ ID NO:3), H3 (LEGLVLTHQQF; SEQ ID NO:4), and H4 (AEIYEAFENIYPILKGFRK; SEQ ID NO:5) are indicated.

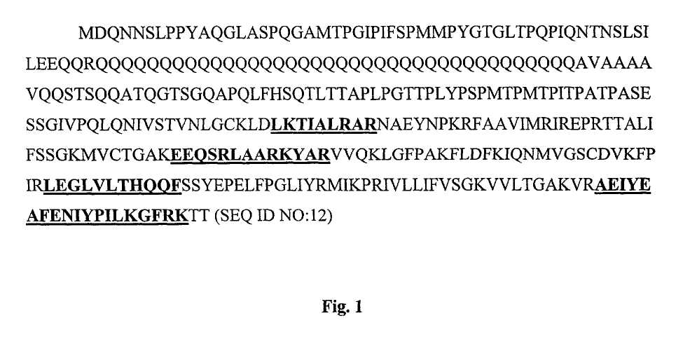

FIG. 2 is a representation of portions of therapeutic agents. The portions can serve as, or as part of, the third domain. The portions shown here are referred to in the text below as H1/H2, H2/H3, and H3/H4 (SEQ ID Nos:6-8, respectively).

FIG. 3 is a summary of aggregation observed following co-transfection of cells with the HD-104Q-EGFP construct (under the control of the EF-1 alpha promoter) and therapeutic agents in which the third domain contained the H1/H2, H2/H3, or H3/H4 sequences (upper Table). The results were collected following two independent transfections, and cells were counted in 6-8 separate fields. Almost identical results were obtained when the promoters driving each expressed polypeptide were switched and the therapeutic agent was tagged with DS Red (lower Table).

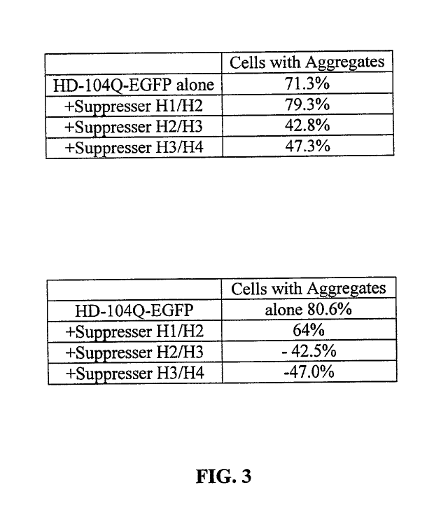

### DETAILED DESCRIPTION

Therapeutic Agents that Inhibit Protein Aggregation

The therapeutic agents of the invention are tri-domain molecules (e.g. tri-peptide fusion proteins) that inhibit interaction (e.g. aggregation, dimerization, or other complex formation) between proteins (e.g. proteins expressed by or within biological cells, such as neurons). An advantage of the invention is the number and type of different proteins whose interaction can be inhibited. These proteins include those with polyglutamine repeat regions, many of which (e.g., the huntingtin protein, atrophin-1, ataxin-1, ataxin-2, ataxin-3, the alphal a-voltage dependent calcium channel, ataxin-7, the androgen receptor, alpha, beta, and gamma syncleins, those involved in amyloidosis, such as those with immunoglobulin light chains, amyloid-associated protein, mutant transthyretin, beta2 microgloblin, beta2 amyloid protein, and the prion proteins) are described below in the context of a screening assay for therapeutic agents.

The therapeutic agents may be, but are not necessarily, symmetrical molecules. That is, a therapeutic agent can be a molecule having identical or substantially identical first and second domains (e.g. polypeptides, other organic molecules, or chemical moieties) separated by a third domain. The first and second domains may also, but need not necessarily, inhibit interaction between identical (or substantially identical) polypeptide targets.

Those of ordinary skill in the art are well able to carry out tests to determine whether protein-protein interaction has been inhibited. One can, for example, carry out the aggregation assay described below (and in Kazantsev et al., Proc. Natl. Acad. Sci. USA 96:11404-11409, 1999). A therapeutic agent of the invention is one that inhibits protein aggregation in that assay by at least 25% (i.e., by 30, 40, 50, 60, 70, 80, or 90% or more). Therapeutic agents can also be assessed by their ability to prolong the time it would ordinarily take proteins to interact. A therapeutic agent of the invention is one that increases the time required for the proteins to interact (e.g., to dimerize or aggregate) by at least 25%. That is, polypeptides may take 50%, 100%, 200%, or more, time to interact in the presence of the therapeutic agent than they would take to interact in its absence.

As indicated above, the first and second domains can consist of any substance that binds to a polypeptide target. More specifically, the first and second domains can include regions rich in glutamine residues, for example, a stretch of consecutive glutamine residues. These stretches of polyQ residues can vary in length from as few as three to as many as 300 glutamine residues. For example, the first and second domains can each contain 3, 7, 10, 20, 30, 37, 38, 39, 40, 50, 75, 100, 150, 200, 250 or 300 glutamine residues. The first and second domains can also contain amino acid residues other than glutamine, so long as the domains bind the target polypeptides. For example, polypeptides consisting of at least 80% glutamine (e.g., 85, 90, 95, or 98% glutamine) are useful domains.

If desired, the therapeutic agent can be made more soluble by inclusion of hydrophilic amino acid residues (e.g. residues of aspartic or glutamic acid). For example, 5, 10, 20 or more residues of either aspartic or glutamic acid, or a mixture of both, can be added to the first, second, or third domain of the therapeutic agent.

The third domain can assume a number of configurations so long as it separates the first and second domains in a way that prevents the bound polypeptide targets (i.e., the polypeptides bound to each of the first and second domains) from interacting as they otherwise would. The third domain can form an essentially straight bridge between the first and second domains or it can include a bend or kink so that the first and second domains are angled with respect to one another. The third domain can include a polypeptide or any other physiologically acceptable polymer. The polypeptide, polymeric structure, or other spacer can be naturally occurring or synthetic. For example, the third domain can include polypeptides having alpha helical (e.g. the H2/H3 and H3/H4 containing polypeptides described in the examples below) or beta-pleated tertiary structures, or can include benzol rings. The benzol rings can separate, for example, a sugar chain containing polyamides.

Physiologically acceptable polymer-based compositions that can be used as the third domain include any of the linking groups described in U.S. Pat. No. 5,830,462. Such groups can be assembled into a chain of 1 to 30, or 1 to 20 non-hydrogen atoms, and can be composed largely or entirely of carbon, hydrogen, nitrogen, oxygen, sulfur and phosphorous. The polymer chain can contain functional moieties, for example, amides, esters, amines, ethers, thioethers, disulfides, and hydrazines, and can be composed of aliphatic, aromatic, alicyclic, and heterocyclic groups. Carbon chains can be synthesized. Some non-limiting examples include: alkylene, azalkylene, arylene, ardialkylene, decylene, octadecylene, azapentylene, 5-azadecylene, N-butylene 5-azanonylene, phenylene, xylylene, p-dipropylenebenzene, and bis-benzoyl 1,8-diaminooctane. The suitability of the linking chain can be tested by, for example, generating a complete tri-domain therapeutic agent with the linking chain as the third domain, and testing the efficacy of the agent in an assay described herein.

In the event the third domain includes a polypeptide, the polypeptide can have random coil, —helical or —pleated tertiary structures. Polypeptides that form suitable flexible linkers are well known in the art (see, e.g., Robinson and Sauer Proc. Natl. Acad. Sci. USA 95:5929-5934, 1998). —helical linkers can impart additional rigidity. An example of a helical linker is provided by Pantoliano et al., Biochem., 30:10117-10125, 1991). Another helical linker is a polypeptide sequence which folds as a coiled-coil, especially a dimeric parallel or anti-parallel coiled-coil. In embodiments in which the first and second domains are identical, the third domain can be one strand of an anti-parallel coiled-coil. The therapeutic molecule in this embodiment is, therefore, a heterodimer. The degree of separation between the first and second domains can be modulated by varying the length of the coiled-coil. Further structural stability can be obtained by employing helical scaffold proteins, such as proteins containing multiple HEAT repeats, armadillo repeats, or tetratricopeptide (TPR) repeats (Groves and Barford, Curr. Opin. Struct. Biol. 9:383-389, 1999). Similarly, —stranded structures can be a component of the third domain. Exemplary —stranded structures include —pleated tertiary structures and—helix proteins (Jenkins et al., J. Struct. Biol. 122:236-246, 1998).

The third domain can also be designed by computer modeling (see, e.g., U.S. Pat. No. 4,946,778). Software for molecular modeling is commercially available from, for example, Molecular Simulations, Inc. The third domain can be optimized to, for example, reduce antigenicity or increase stability by using standard mutagenesis techniques and appropriate biophysical tests, as practiced in the art of protein engineering, and functional assays, as described herein.

As described above, in some instances, the therapeutic agent will consist, wholly or partially, of a polypeptide. As used herein, “polypeptide” means any peptide-linked chain of amino acid residues, regardless of length or post-translational modification. The terms “polypeptide,” “peptide,” and “protein” are interchangeable.

Nucleic Acid Molecules Can Encode Tri-Domain Therapeutic Agents

The invention also features isolated DNA molecules that encode therapeutic agents having three domains. Generally, nucleic acids are within the invention so long as they encode any of the therapeutic agents that contain the first, second, and third polypeptide domains described herein. For example, the nucleic acid molecules can encode a polypeptide having a first domain that binds a first protein that has at least seven consecutive glutamine residues (e.g., 7, 10, 15, 20, 25, 30, 35, 36, 37, 38, 39, or 40 or more consecutive glutamine residues); a second domain that binds a second protein that has at least seven consecutive glutamine residues (e.g., 7, 10, 15, 20, 25, 30, 35, 36, 37, 38, 39, or 40 or more consecutive glutamine residues); and a third domain that separates the first domain from the second domain.

An “isolated” nucleic acid molecule is one that is free from the sequence that flanks it in a naturally occurring cell or organism (i.e., one that has not be genetically modified). The term therefore includes a recombinant DNA incorporated into a vector, into an autonomously replicating plasmid or virus, or into the genomic DNA of a prokaryote or eukaryote. It also includes a separate molecule such as a cDNA, a genomic fragment, a fragment produced by polymerase chain reaction (PCR), or a restriction fragment. It also includes a recombinant nucleotide sequence that is part of a hybrid gene, i.e., a gene encoding a fusion protein.

The isolated nucleic acid molecules of the invention can be placed into an expression vector, and that vector can be introduced into a biological cell, where the nucleic acid molecule will be expressed as a therapeutic agent. Accordingly, expression vectors and isolated or purified cells that contain the nucleic acid molecules described herein are within the scope of the present invention.

One of ordinary skill in the art is well able to construct and express the nucleic acid molecules and vectors that encode tri-domain therapeutic agents. For example, it is well known that nucleic acid molecules can be placed under the control of one or more expression control sequences such as transcriptional promoters, enhancers, suitable mRNA ribosomal binding sites, and sequences that terminate transcription and translation. If guidance is required, those of ordinary skill in the art can consult one of the many technical manuals available, such as Sambrook et al., (Cloning—A Laboratory Manual, 2nd Edition, Cold Spring Harbor Laboratory Press, Cold Spring Harbor, N.Y.).

Vectors useful in this invention include plasmid vectors and viral vectors. Viral vectors can be those derived from retroviruses, adenovirus, adeno-associated virus, SV40 virus, or herpes viruses. Once introduced into a host cell (e.g., bacterial cell, yeast cell, avian cell, mammalian cell), the vector can remain episomal, or be incorporated into the genome of the host cell.

In bacterial systems, a number of expression vectors may be advantageously selected depending upon the use intended for the gene product being expressed. For example, when a large quantity of such a protein is to be produced, e.g., for raising antibodies to the polypeptide, a vector capable of directing the expression of high levels of a fusion protein (e.g., a GST fusion protein) that is readily purified may be desirable. In general, such fusion proteins are soluble and can easily be purified from lysed cells by adsorption to, e.g., glutathione-agarose beads followed by elution in the presence of free glutathione.

In order to circumvent difficulties associated with the propagation of long CAG repeats in bacteria, a cloning strategy has been developed which used alternating CAG/CAA repeats encoding glutamine residues (for example, the 25 residues that can be found in the huntingtin gene of healthy individuals, the 104 residues present in pathological in HD, and stretches of polyQ elongated beyond pathological range (e.g. 191, 230, or 300 glutamine residues)). More specifically, CAACAGCAGCAACAGCAA (SEQ ID NO:9) and complementary TTGTTGCTGTTGCTGCTG (SEQ ID NO:10) oligonucleotides were annealed to generate double strand duplex DNA with trinucleotide extensions. Short duplex DNA molecules were used as starting material for two consecutive ligations to obtain alternating CAG/CAA repeats (CAACAGCAGCAACAGCAA)n (SEQ ID NO:9) of different lengths. The second ligation reaction was terminated by addition of dsDNA linkers that included 5′ trinucleotide extensions and the restriction sites HindIII at the 5′ end and PstI at the 3′ end with respect to the CAG/CAA DNA strand. Alternating CAG/CAA repeats of different lengths were subcloned into Bluescript-KS vector and maintained in XL-1 Blue (Stratagene). Repetitive CAACAGCAGCAACAGCAA (SEQ ID NO:9) consensus was verified by two-strand sequence analyses, and clones containing 25-300 alternating CAG/CAA repeats were selected to generate mammalian expression constructs (see Kazantsev et al., Proc. Natl. Acad. Sci. USA 96:11404-11409, 1999).

Screening for Therapeutic Agents that Inhibit Protein Aggregation

Therapeutic Agents containing the three domains described above can be assessed in a number of in vitro or in vivo assays known to those of ordinary skill in the art, including those described in the examples below. For example, therapeutic agents can be assessed in the fluorescence-based assay described by Kazantsev et al. (Proc. Natl. Acad. Sci. USA 96:11404-11409,1999) and in U.S. Ser. No. 09/405,048, which are both incorporated herein by reference in their entirety.

The fluorescence-based assay exploits the detergent resistance of polyglutamine aggregates and will facilitate high-throughput screening for agents that suppress polyglutamine aggregation in cells. The assay is based on the discovery that polyglutamines of normal length can form insoluble detergent-resistant aggregates when coexpressed with extended polyQ tracts. Once the process of aggregation is initiated, an expanded length of the polyQ tract is no longer required for joining the aggregate. Thus, the depletion from the cell pool by sequestration in aggregates of any protein with a significant polyQ segment may represent a potential mechanism for the cellular toxicity of polyQ aggregates.

The assay described by Kazantsev et al. (supra) can be carried out by, for example, providing a first polypeptide, which is labeled with a detection moiety (e.g., an enzyme or fluorescent protein (see below)) that is inactive in the presence of a denaturant and a second polypeptide (which may or may not be identical to the first); forming a mixture by contacting the first and second polypeptides with a test compound; adding a denaturant to the mixture; and determining the activity of the detection moiety. Of course, one of ordinary skill in the art will recognize and conduct control experiments, such as experiments in which the test compound is omitted or added in an inactive state. A decrease in activity following addition of the denaturant indicates that the test compound has prevented at least some of the polypeptides from aggregating, thereby leaving them susceptible to inactivation by the denaturant. Thus, a loss of the signal generated by the detection moiety indicates that the putative therapeutic agent has successfully prevented at least some aggregation between the first and second polypeptides. The first or second polypeptide can be immobilized, or both can be in solution or within a cell.

Polypeptides that can be used in aggregation assays can be naturally occurring or non-naturally occurring. Protein database searches reveal that hundreds of polyQ-containing proteins have been identified to date (Kazantsev et al., Proc. Natl. Acad. Sci. USA 96:11404-11409, 1999). An interesting class of nuclear proteins that contain glutamine-rich regions and often homopolymeric glutamine stretches are transcription factors and transcriptional coactivators (Kazantsev et al., Proc. Natl. Acad. Sci. 96:11404-11409, 1999). Accordingly, therapeutic agents of the invention can be agents that prevent the aggregation (or other physical interaction, such as dimerization) of these factors and coactivators

Useful polypeptides include those that contain regions rich in glutamine residues (e.g., consecutive glutamine residues). Polypeptides that contain stretches of polyglutamine include the huntingtin protein (which causes Huntington's Disease), atrophin-l (which causes dentatorubralpallidoluysian atrophy), ataxin-1(which causes spinocerebellar ataxia type 1), ataxin-2 (which causes spinocerebellar ataxia type 2), ataxin-3 (which causes spinocerebellar ataxia type 3), the alphala-voltage dependent calcium channel, ataxin-7, and the androgen receptor (which causes spinobulbar muscular atrophy). Other naturally occuring polypeptides that aggregate with one another include alpha, beta, and gamma syncleins, which are encoded by three genes and are abundantly expressed in neurons. Syncleins have been implicated in Alzheimer's Disease (AD), Parkinson's Disease (PD), and breast cancer. Other useful polypeptides include those involved in amyloidosis, such as those with immunoglobulin light chains (which are involved in multiple myeloma and various other B cell proliferative disorders), amyloid-associated protein, mutant transthyretin (which is involved in familial amyloid polyneuropathies), beta2 microgloblin (which is involved in chronic renal dialysis), and beta2 amyloid protein (which is involved in AD). Yet another useful class of proteins is the class of prions. Aggregation of prion proteins causes spongiform encephalopathies such as Creutzfeldt-Jakob disease and kuru in humans (and counterpart diseases in livestock, such as “mad cow disease”). The therapeutic agents of the invention are useful in inhibiting interaction between any of these proteins and, thus, are useful in treating any of the aforementioned disorders.

As stated above, the “first” polypeptide is detectably labeled. Examples of detectable labels include various enzymes, prosthetic groups, fluorescent materials, luminescent materials, bioluminescent materials, and radioactive materials. Examples of suitable enzymes include horseradish peroxidase, alkaline phosphatase, -galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, green fluorescent protein (or enhanced green fluorescent protein) and phycoerythrin; an example of a luminescent material is luminol; examples of bioluminescent materials include luciferase, luciferin, and aequorin; and examples of suitable radioactive materials include ¹²⁵I, ¹³¹I, ³⁵S, ³²P, and ³H.

Test compounds that can be screened in accordance with the invention include polypeptides, antibodies (wherein the antibody is present as either the first or second domain, or both), and monomeric organic compounds (i.e., “small molecules,” which may occupy the position of the first or second domain, or both).

If desired, preliminary tests can be performed to determine whether or not a substance (e.g., a polypeptide or small molecule) binds to a protein of interest (e.g., the huntingtin protein). If it does, it is suitable for inclusion in a putative therapeutic agent as a first or second domain. Determining whether an agent will bind a protein of interest is a matter of routine experimentation. For example, a test substance can be incubated with an epitope-tagged protein of interest. Display libraries can also be used to identify substances that bind to proteins of interest (e.g. polyglutamine-containing proteins). In this approach, test peptides are displayed on the surface of a cell or viral particle, and the ability of particular cells or viral particles to bind an appropriate protein (e.g., huntingtin, alpha, beta, and gamma syncleins, atrophin-1, and ataxin-1, -2, or -3) via the displayed product can be detected in a “panning assay” (Ladner et al., WO 88/06630).

Conditions Amenable to Treatment

Huntington's disease (HD) is an autosomal dominant and progressive neurodegenerative disorder. It is associated with selective neuronal cell death that occurs primarily in the cortex and striatum and is characterized by a movement disorder, cognitive deficits, and psychiatric symptoms. HD is caused by an expansion of a CAG codon repeat in the first exon of the huntingtin (htt) gene, which encodes a 350 kDa protein of unknown function (Ambrose et al., Somat. Cell Mol. Genet. 20:27-38, 1994). The nucleotide triplet CAG encodes the amino acid glutamine (“Gln” or “Q”). Thus, CAG repeats encode polyglutamine regions within huntingtin (and wherever they occur). The polyglutamine region of huntingtin from non-HD individuals contains about 8-31 consecutive glutamine residues. When the protein has more then 37 consecutive glutamine residues, mild to severe HD results. The more severe cases of the disease exhibit up to about 68 glutamine residues. A juvenile onset form of HD is characterized by more widespread neuronal degeneration and is caused by expansions above approximately 65 repeats.

In addition to HD, at least seven other inherited neurodegenerative disorders are associated with CAG expansions. Increasing the length of CAG repeats in the coding regions of unrelated genes, and resulting polyglutamine regions in the encoded proteins, causes a similar pattern of neuron degeneration, indicating a similar, if not identical, mechanism of cell death. This cell death may be caused by abnormal protein-protein interactions mediated by elongated polyglutamines. Thus, each of the neurodegenerative disorders associated with CAG expansions are amenable to treatment with the therapeutic agents of the present invention.

The therapeutic agents of the invention are not, however, limited to the treatment of neurodegenerative disorders. The therapeutic agents of the invention can be used to treat any disease, disorder, or condition that results from an abnormal or undesirable association between two polypeptides (like or unlike). For example, the therapeutic agents of the invention can be used to treat Alzheimer's disease (by inhibiting the association of tau proteins) and disorders in which polyglutamine-containing transcription factors or coactivators are undesirably active (e.g. disorders (e.g., cancers) associated with homodimerization of jun or hexamerization of p53).

“Treatment” encompasses administration of a therapeutic agent as a prophylactic measure to prevent the occurrence of disease or to lessen the severity or duration of the symptoms associated with the disease. Physicians and others of ordinary skill in the art routinely make determinations as to the success or failure of a treatment. Treatment can be deemed successful despite the fact that not every symptom of the disease is totally eradicated.

Animal Models

It is usual in the course of developing a therapeutic agent that tests of that agent in vitro or in cell culture are followed by tests in animal models of human disease, and further, by clinical trials for safety and efficacy in humans. Accepted animal models for many diseases are now known to those of ordinary skill in the art. For example, therapeutic agents of the present invention can be screened in the Drosophila model of neurodegeneration presented in Example 2.

Mammalian models for Huntington's disease are also available. To generate these models, the Hd protein homolog is first cloned from the genome of the selected mammal using standard techniques. For example, the sequence can be amplified by PCR or obtained by screening an appropriate library under conditions of low stringency (as described, e.g., in Sambrook et al. supra.). Subsequently, CAG repeats are introduced into the HD gene by molecular cloning and mutagenesis techniques. The site for insertion of the repeat sequence can be located by alignment of the HD cDNA from the desired mammal with the human cDNA for HD. The modified HD gene with artificially expanded repeats is reintroduced into the mammal using standard methods for transgenesis.

If the desired animal model is a mouse, numerous models of HD are available (see, e.g., U.S. Pat. No. 5,849,995; for a review, see Chicurel et al., Expression of Huntington 's Disease Mutation in Mice at http://www.hdfoundation.org/PDF/hdmicetable.pdf) (2000). The mouse HD cDNA sequence is deposited in GenBank as L23312 and L23313. Methods for generating transgenic mice are routine in the art (See, e.g., Hogan et al., Manipulating the Mouse Embryo, Cold Spring Harbor Laboratory Press, Cold Spring Harbor, N.Y. (1 994)). A mouse bearing a transgene comprising the HD gene and expanded CAG repeats has symptoms similar to the human disease. Murine symptoms can include hyperactivity, circling, abnormal gait, tremors, learning deficits, hypoactivity, and hypokinesis. Neuropathological symptoms include general brain atrophy, progressive striatal atrophy, neuropil aggregates, inclusions in the striatum, reduced dendritic spines, cell loss in the cortex, and striatum. Any of these behavioral or physiological deficits can be assessed in order to determine the efficacy of a given therapeutic agent of the invention. For example, the agent can be administered to a transgenic mouse model, generated as described above. The symptoms of a treated mouse can be compared to untreated mice at various times during and after treatment. In addition, treated and untreated mice can be sacrificed at various intervals after treatment, and the neuropathology of the brain can be analyzed. Thus, the efficacy of the treatment can be evaluated readily by comparing the behavioral symptoms, neuropathological symptoms, and clinical symptoms of treated and untreated mice.

Administration

The therapeutic agents of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well known reference text in this field, and in the USP/NF (United States Pharmacopeia and the National Formularly).

A pharmaceutical composition (e.g., a composition containing a therapeutic agent or the DNA molecule encoding it) is formulated to be compatible with its intended route of administration. Examples of routes of administration include oral, rectal, and parenteral, for example, intravenous, intradermal, and subcutaneous, transdermal (topical), and transmucosal, administration.

In treating neurodegenerative disorders, or other disorders within the central nervous system, with therapeutic agents of the invention, the agents must contact the affected neurons (e.g., neurons of the cortex and striatum) to provide a therapeutic effect. If the agents are provided orally or parenterally (e.g., intravenously), rather than locally, the agents must be either permeable to the blood-brain barrier or be assisted in traversing it.

The blood-brain barrier is an obstacle for the delivery of drugs from circulation in the bloodstream to the brain. The endothelial cells of brain capillaries are connected by tight intercellular junctions, which inhibit the passive movement of compounds out of the blood plasma into the brain. These cells also have reduced pinocytic vescicles in order to restrict the indiscriminate transport of materials intracellularly. These features of the brain regulate the exchange of materials between plasma and the central nervous system. Both active and passive transport mechanisms operate to exclude certain molecules from traversing the barrier. For example, lipophilic compounds are more permeable to the barrier than hydrophilic compounds (Goldstein et al., Scientific American 255:74-83, 1996; Pardridge et al., Endrocrin. Rev. 7:314-330, 1996).

However, the blood-brain barrier must also allow for the selective transport of desired materials into the brain in order to nourish the central nervous system and to remove waste products. The mechanisms by which this is accomplished can provide the means for supplying the therapeutic agents described herein.

Therapeutic agents of the invention can be delivered to the CNS following conjugation with other compounds as follows (and as described further in U.S. Pat. No. 5,994,392). In one instance, polar groups on a drug are masked to generate a derivative with enhanced lipophilic qualities. For example, norepinephrine and dopamine have been modified with diacetyl and triacetyl esters to mask hydroxyl groups. An implementation of this strategy has been previously used to create a pro-drug derivative of dopamine (see U.S. Pat. No. 5,994,392). The modified drugs are generally referred to as pro-drugs. This method has the additional advantage of providing an inactive species of the drug in general circulation. Thus, the pro-drug is able to cross the blood-brain barrier. Subsequently, enzymes present in the central nervous system are able to hydrolyze the ester linkages, thereby liberating the active drug. Thus, therapeutic agents of the invention are chemically modified to create pro-drugs by, e.g., conjugation to a lipophilic moiety or carrier. A compound or derivative thereof, having at least one free hydroxyl or amino group, can be coupled to a desired carrier. The carrier can be a fatty acid, a steroid, or another lipophilic moiety.

For example, the hydroxyl groups are first protected with acetonide. The protected agent is then reacted with the desired carrier in the presence of a water-extracting compound (e.g., dicyclohexyl carbodiiamide), in a solvent (e.g., dioxane, tetrahydrofurane), or N,N dimethylformamide at room temperature. The solvent is then removed, and the product is extracted using methods routinely used by those of ordinary skill in the art. Amine groups can be coupled to a carboxyl group in the desired carrier. An amide bond is formed with an acid chloride or low carbon ester derivative of the carrier. Bond formation is accompanied by HCl and alcohol liberation. Alcohol groups on the drug compound can be coupled to a desired carrier using ester bonds by forming an anhydride derivative, i.e. the acid chloride derivative, of the carrier. One of ordinary skill in the art of chemistry will recognize that phophoramide, sulfate, sulfonate, phosphate, and urethane couplings are also useful for coupling a therapeutic agent to a desired carrier.

Procedures for delivering therapeutic agents of the invention to the CNS can also be carried out using the transferrin receptor as described, for example, in U.S. Pat. No. 6,015,555. To implement this procedure, the agents are conjugated to a molecule that specifically binds to the transferrin receptor (e.g., an antibody or fragment thereof, or transferring. Methods for obtaining antibodies against the transferrin receptor and for coupling the antibodies to a desired drug are also described in U.S. Pat. No. 6,015,555.

Monoclonal antibodies that specifically bind to the transferrin receptor include OX-26, T58/30, and B3/25 (Omary et a., Nature 286:888-891, 1980), T56/14 (Gatter et al., J. Clin. Path. 36:539-545, 1983), OKT-9 (Sutherland et al., Proc. Natl. Acad. Sci. USA 78:4515-4519, 1981), L5.1 (Rovera, Blood 59:671-678, 1982) and 5E-9 (Haynes et al., J. Immunol. 127:347-351, 1981). In one embodiment, the monoclonal antibody OX-26 is used. The antibody of choice can be an Fab fragment, a F(ab')₂ fragment, a humanized antibody, a chimeric antibody, or a single chain antibody.

The antibody to the transferrin receptor is conjugated to a desired therapeutic agent of the invention with either a cleavable, or non-cleavable linker. The preferred type of linker can be determined without undue experimentation by making cleavable and non-cleavable conjugates and assaying their activity in, for example, an in vitro or cell culture assay described herein. Examples of chemical systems for generating non-cleavable linkers include the carbodiimmide, periodate, sulfhydryl-maleimide, and N-succinimidyl-3-(2-puridyldithio) propionate (SPDP) systems. Carbodiimide activates carboxylic acid groups, which then react with an amino group to generate a noncleavable amide bond. This reaction is useful for coupling two proteins. Periodate is used to activate an aldehyde on an oligocacharide group such that it can react with an amino group to generate a stable conjugate. Alternatively, a hydrazide derivative of the desired compound can be reacted with the antibody oxidized with periodate. Sulfhiydryl-maleimide and SDPD use sulfhydryl chemistry to generate non-cleavable bonds. SDPD is a heterobifunctional crosslinker that introduces thiol-reactive groups. In the sulfhydryl-maleimide system, an NHS ester (e.g., gamma-maleimidobutyric acid NHS ester) is used to generate maleimide derivative, for example, of a protein drug or antibody. The maleimide derivative can react with a free sulfhydryl group on the other molecule.

Cleavable linkers are also useful. Cleavable linkers include acid labile linkers such as cis-aconitic acid, cis-carboxylic alkadienes, cis-carboxylic alkatrienes, and polypeptide-maleic anhydrides (see U.S. Pat. No. 5,144,011).

In a preferred embodiment, the therapeutic agent of the invention is a tri-domain polypeptide. Such a polypeptide can be covalently attached to an antibody specific for the transferrin receptor. The coupling can be made by fusing a gene encoding the therapeutic polypeptide to a gene encoding a monoclonal antibody specific for the transferrin receptor. The gene encoding the monoclonal antibody can obtained using polymerase chain reaction strategies to amplify the gene from hybridoma cells (see, e.g., Orlandi et al., Proc. Natl. Acad. Sci. USA 86:3833-3837, 1989; Larrick et al., Bio/technology 7:934-938, 1989; Gavilondo et al., Hybridoma 9:407-417, 1990). PCR primers are designed to anneal to the leader sequence, or the first framework region of the antibody variable domain and to the J region or the constant region. The PCR primers are used to amplify the immunoglobulin gene from cDNA or genomic DNA of the hybridoma. The amplification product is cloned into an expression vector or a cloning vector. The antibody can also be humanized, modified to be a single chain antibody, a chimera, or other derivatives known in the art. In one embodiment, construction of a single chain antibody is preferred in order to facilitate covalent fusion with the polypeptide agent, for example, a tri-domain therapeutic polypeptide.

The cloned antibody gene is fused to a covalent linker attached to the polypeptide agent of the invention. These features can be inserted using synthetic oligonucleotides, standard cloning procedures, and PCR. They can be amino- or carboxy- terminal to the antibody gene. Oligonucleotides and restriction sites for cloning are selected such that the linker and the desired polypeptide compound are inserted in frame with respect to the antibody coding sequence. Moreover, a protease recognition site can be included in the linker if cleavage of the antibody is required after delivery. The resulting fusion gene can be inserted into an expression vector as appropriate, and the fusion protein can produced, for example, in E. coli, insect cells, and mammalian cells in tissue culture. Alternatively, the fusion gene can be inserted into a gene therapy vector for expression in a subject.

Therapeutic polypeptides can also be modified by lipidation in order to stabilize the polypeptide and to promote traversal of the blood-brain barrier. A method for lipidation of antibodies is described by Cruikshank et al. (J. Acquired Immune Deficiency Syndromes and Human Retrovirology 14:193, 1997).

The efficacy of strategies to deliver a desired compound across the blood-brain barrier can, of course, be monitored. The desired agent, conjugated for delivery across the blood-brain barrier, is administered to a test mammal (e.g., a rat, a mouse, a non-human primate, a cow, a dog, a rabbit, a cat, or a sheep). One of ordinary skill in the art will, however, recognize that the permeability of the blood-brain barrier varies from species to species, with the human blood-brain barrier being the least permeable. The mode of administration can be the same as the desired mode of treatment, or it can be intravenous. For a comprehensive analysis, a set of test mammals is used. The test mammals are sacrificed at various times after the agent is administered and are then perfused through the heart with Dulbecco's phosphate-buffered saline (DPBS) to clear the blood from all organs. The brain is removed, frozen in liquid nitrogen, and subsequently sectioned in a cryostat. The sections are placed on glass microscope slides. The presence of the desired agent is then detected in the section, for example with an antibody, or by having administered a radiolabeled or otherwise tagged compound (such labeled polypeptide agents are described above). Detection is indicative of the compound having successfully traversed the blood-brain barrier. If a method of enhancing the compounds permeability to the blood-brain barrier is being assessed, then the amount of the agent detected in a brain section can be compared to the amount detected in a brain section from an animal treated with the same compound without the enhancing method.

The terms “blood-brain barrier permeant” or “blood-brain barrier permeable” are qualities of a compound for which the ratio of a compound's distribution at equilibrium in the cerebrospinal fluid (CSF) relative to its distribution in the plasma (CSF/plasma ratio) is greater than 0.01, generally at least 0.02, preferably at least 0.05, and most preferably at least 0.1.

One aspect of the invention, described above, provides for isolated DNA molecules that encode a tri-domain therapeutic polypeptide. These isolated DNA molecules can be inserted into a variety of DNA constructs and vectors for the purposes of gene therapy. As used herein, a “vector” is a nucleic acid molecule competent to transport another nucleic acid molecule to which it has been covalently linked. Vectors include plasmids, cosmids, artificial chromosomes, and viral elements. The vector can be competent to replicate in a host cell or to integrate into a host DNA. Viral vectors include, for example, replication defective retroviruses, adenoviruses and adeno-associated viruses. A gene therapy vector is a vector designed for administration to a subject, for example, a mammal (such as a human), such that a cell of the subject is able to express a therapeutic gene contained in the vector.

The gene therapy vector can contain regulatory elements (e.g., a 5′ regulatory element, an enhancer, a promoter, a 5′ untranslated region, a signal sequence, a 3′ untranslated region, a polyadenylation site, and a 3′ regulatory region). For example, the 5′ regulatory element, enhancer, or promoter can regulate transcription of the DNA encoding the therapeutic polypeptide. The regulation can be tissue specific. For example, the regulation can restrict transcription of the desired gene to brain cells (e.g., cortical neurons or glial cells); hematopoietic cells; or endothelial cells. Alternatively, regulatory elements can be included that respond to an exogenous drug, for example, a steroid, tetracycline, or the like. Thus, the level and timing of expression of the therapeutic polypeptide can be controlled.

Gene therapy vectors can be prepared for delivery as naked nucleic acid, as a component of a virus, or of an inactivated virus, or as the contents of a liposome or other delivery vehicle. Alternatively, the gene delivery agent, for example, a viral vector, can be produced from recombinant cells that produce the gene delivery system. Appropriate viral vectors include retroviruses, for example, Moloney retrovirus, adenoviruses, adeno-associated viruses, and lentiviruses, for example, Herpes simplex viruses (HSV). HSV is potentially useful for infecting nervous system cells.

A gene therapy vector can be administered to a subject, for example, by intravenous injection, by local administration (see U.S. Pat. No. 5,328,470) or by stereotactic injection (see e.g., Chen et al., Proc. Natl. Acad. Sci. USA 91:3054-3057, 1994). The gene therapy agent can be further formulated, for example, to delay or prolong the release of the agent by means of a slow release matrix. One preferred method of providing a recombinant therapeutic tri-domain polypeptide, is by inserting a gene therapy vector into bone marrow cells harvested from a subject. The cells are infected, for example, with a retroviral gene therapy vector, and grown in culture. Meanwhile, the subject is irradiated to deplete the subject of bone marrow cells. The bone marrow of the subject is then replenished with the infected culture cells. The subject is monitored for recovery and for production of the therapeutic polypeptide.

An appropriate dosage of the therapeutic agents of the invention must be determined. An effective amount of a tri-domain molecule is the amount or dose required to ameliorate a symptom of a disorder associated with trinucleotide repeat expansion, Alzheimer's disease, or cancers associated with the dimerization or other association of transcriptional regulators. Determining the amount required to treat a subject is routine to one of ordinary skill in the art (e.g., a physician, pharmacist, or researcher). First, the toxicity and therapeutic efficacy of an agent (i.e. a tri-domain molecule) is determined. Routine protocols are available for determining the LD₅₀ (the dose lethal to 50% of the population) and the ED₅₀ (the dose therapeutically effective in 50% of the population) in non-human animals. The therapeutic index is measured as the ratio of the LD₅₀/ED₅₀. Compounds, formulations, and methods of administration with high therapeutic indices are preferable as such treatments have little toxicity at dosages that provide high efficacy. Compounds with toxic or undesirable side effects can be used, if means are available to deliver the compound to the affected tissue, while minimizing damage to unaffected tissue.

In formulating a dosage range for use in humans, the effective does of tri-domain compound can be estimated from in vitro cell studies and in vivo studies with animal models. If an effective dose is determine for ameliorating a symptom in cell culture, a dose can be formulated in an animal in order to achieve a circulating plasma concentration of sodium butyrate that falls in this range. An exemplary dose produces a plasma concentration that exceeds the IC₅₀ (i.e., the concentration of the test compound which achieves a half-maximal inhibition of symptoms) as determined in cell culture assays. The circulating plasma concentration can be determined, for example, by administering a labeled therapeutic composition to the test animal, obtaining a blood sample, and quantitating the amount of labeled compound present at various times after administration.

An appropriate daily dose of a tri-domain therapeutic can be between about 0.1 mg/kg of body weight to about 500 mg/kg, or between about 1 mg/kg to about 100 mg/kg. The dose can be adjusted in accordance with the blood-brain barrier permeability of the compound. For example, a polypeptide such as an antibody can be administered at a dosage of 50 mg/kg to 100 mg/kg in order to treat the brain. The dose for a patient can be optimized while the patient is under care of a physician, pharmacist, or researcher. For example, a relatively low dose of a tri-domain therapeutic can be administered initially. The patient can be monitored for symptoms of the disorder being treated (e.g., HD). The dose can be increased until an appropriate response is obtained. In addition, the specific dose level for any particular subject can vary depending on the age, body weight, general health, gender, and diet of the subject, the time of administration, the route of administration, the rate of excretion, and other drugs provided in combination.

The efficacy of a dose of any therapeutic agent can be determined in a subject. For example, the subject can be monitored for clinical symptoms, for example, a symptom of a trinucleotide repeat disease, such as a symptom of HD. Behavioral symptoms of HD include irritability, apathy, lethargy, depression, hostile outbursts, loss of memory and/or judgment, loss of ability to concentrate, anxiety, slurred speech, difficulty swallowing and/or eating, and inability to recognize persons. Clinical symptoms of HD include loss of coordination, loss of balance, inability to walk, uncontrolled movements of the fingers, feet, face, and/or trunk, rapid twitching, tremors, chorea, rigidity, and akinesia (severe rigidity).

### EXAMPLES

### Example 1

### Suppression of Protein Aggregation in vitro

Mammalian expression constructs: The polyglutamine expression constructs used in this study were described in detail in Kazantsev et al. (Proc. Natl. Acad. Sci. USA 96:11404-11409, 1999). Spacers (referred to herein as “the third domain”) containing two alpha-helical domains were amplified by PCR from full length TBP (Genbank Accession No. M55654), as described in Kazantsev et al. (supra). Wild type TBP cDNA (Genbank Accession No. M55654) was amplified from genomic DNA extracted from eHeLa cells, again, as described in Kazantsev et al. (supra). Desired TBP fragments were amplified by PCR with primers that introduced novel KpnI and BglII restriction sites in the N-terminus and novel HindIII and BamHII restriction sites in the C-terminus. HD25Qmyc plasmid was used as a template. DNA fragments encoding H1/H2, H2/H3, and H3/H4 sequences were digested with KpnI and HindIII subcloned in front of 25Q in BlueScript vector. DNA fragments encoding HD25Q were isolated by digestion with KpnI and BamHI and ligated in front of polypeptide fusion H/H 25Q digested with KpnI and BglII. Resulting HD 25Q H/H 25Q polypeptide fusions were subcloned into a mammalian expression vector (pcDNA 3.1; Invitrogen), using KpnI and BamHI. Final clones were sequenced to verify the accuracy of molecular cloning manipulations. The mammalian expression vector, pBudC4 (Invitrogen) was also used. This vector permits expression of two polypeptides from the same plasmid.

Fluorescent analyses of transfected cells: Polyglutamine aggregation was assayed in Cos-1, Cos-7, NIH 3T3, 293, EcR-293, eHeLa, NT-2, and PC-12 cell lines. Cells were grown on coverslips to 50% confluence and lipofected for two hours with Transfectam reagent (Promega). Polyglutamine aggregation was assayed from 16 to 72 hours after transfection. Cells were fixed in 2% formaldehyde/0.1% triton-X100 for 10 minutes and incubated with primary mouse monoclonal anti-c-myc (Invitrogen) antibody (1: 500) and secondary FluoroLink Cy3 (Amersham Life Science) antibody (1: 2000). Epifluorescent microscopy was performed on a Zeiss Axioplan II equipped with a Quantix CCD camera (Photometrics) and Spectrum imaging software (Scanalytics).

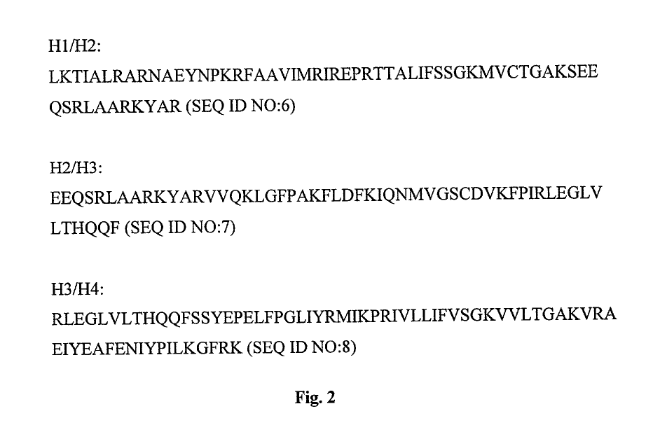

To test the ability of a therapeutic agent to alter aggregation in cell culture, Cos1 cells were transiently transfected with a single plasmid, pBudC4, encoding both an expanded polyglutamine repeat protein, 103QE or F103QE, and either H1/H2, H2/H3 or H3/H4 (FIG. 2). In this system, the 103QE and suppressor protein are expressed in a 1:1 ratio. 103QE is comprised of the first 17 amino acids of huntingtin, followed by a polyQ repeat of 103Qs and epitope tagged at the carboxy terminus with enhanced green fluorescent protein (EGFP). F103QE is identical with the addition of an amino terminal FLAG epitope tag. When transiently transfected in mammalian cells, 103QE rapidly forms aggregates in the majority of cells. Quantitation reveals that expression of 103QE produces aggregates in 71.3% of cells when expressed under the control of the EF-1 α promoter and F103QE produces aggregates in 80.6% of cells when expressed under the control of a CMV promoter. Suppressor peptides were co-expressed with 103Q containing protein, either under the control of the EF1α promoter (with F103QE expression driven by the CMV promoter) or in the opposite orientation, under the control of the CMV promoter (with 103QE expression driven by the EF-1α promoter). A steady reduction in the formation of aggregates in cells following co-transfection with H2/H3 and H3/H4 was observed. Quantitation of cells containing aggregates indicates that all three suppressors inhibit aggregation, with the strongest inhibition mediated by H2/H3 (40% and 47.3%, respectively) and H3/H4 (33.6% and 41.7%, respectively). H1/H2 also inhibited aggregation to a lesser extent when driven by the EF-1α promoter (20.6%), but had no effect when expressed by the CMV promoter.

Not only were the number of cells containing aggregated proteins reduced, but the composition of the aggregates remaining was altered as well. High magnification fluorescence microscopy revealed that co-expression of 103Q proteins with suppressor H2/H3 caused formation of either a single large aggregate or multiple small aggregates, which stained for the therapeutic agent. This co-aggregation of suppressor, as visualized using c-myc antibody with 108Q protein, was observed for each suppressor, regardless of their ability to reduce the number of cells containing aggregates. In cells that continue to produce aggregates, up to 40% contained multi-aggregates as opposed to single inclusions. This failure to form large, single inclusions is similar to what is observed in cells expressing protein with 47Qs, suggesting that H2/H3 interferes with progression of aggregation to a level more similar to less severe disease. In cells co-expressing 103QE and H3/H4, a single aggregate in cells continuing to produce inclusions was the predominant species observed.

Previously, aggregates that formed in mammalian cells by expanded polyglutamine repeat proteins were shown to be resistant to treatment with SDS and to remain insoluble. In the presence of either H1/H2 or H3/H4, but not H2/H3 (FIG. 2), 15% of the co-aggregates that remain reveal a loose structure following treatment with SDS, suggesting that the suppressor alters the composition of the aggregate and loosens the structure. It appears that this property is restricted to large, single aggregates as opposed to the small multiple aggregates, which is consistent with the notion that the suppressors disrupt formation of larger aggregates with a range of effectiveness that may be indicative of a kinetic effect.

To examine the kinetics of aggregation in the presence and absence of suppressor proteins, high resolution video microscopy was performed. Filming live cells permitted the identification of key steps in the aggregation process. After a slow nucleation step, aggregation proceeds rapidly, and normally appears to be complete within 20 minutes. Extended polyglutamines expressed throughout the cell body aggregated, typically via a single “seed”, within forty minutes. Both suppressors delayed the initial nucleation event by 24 hours, when the first aggregates typically appeared in cells expressing 103Q protein alone. Even when multiple seeds did form, extended polyglutamines did not polymerize further into large inclusions, at least within 50 minutes.

Suppressor peptides interact in vitro with polyglutamine containing proteins in a polyQ dependent manner: The theoretical mechanism for the ability of the suppressor protein to inhibit aggregation when co-expressed with an expanded repeat containing portion of the htt protein involves a direct interaction between the two proteins. To investigate whether the amino terminal region of htt interacts in vitro with suppressor proteins, glutathione- S-transferase (GST) pull-down assays were performed. GST-htt fusion proteins, with 20, 51 or 93 polyglutamine repeats, were expressed in bacteria and purified using glutathione-agarose beads. Following incubation with ³⁵S-labeled suppressor proteins (H2/H3 and H3/H4) and subsequent washes, bound protein was visualized by SDS gel electrophoresis and autoradiography. Both H2/H3 and H3/H4 bind to GST-htt fusion proteins. H2/H3 demonstrates the strongest binding, and both peptides bind to GST-htt proteins in a polyQ length dependent manner. The therapeutic agent shows binding to soluble protein as well as co-aggregation with expanded polyglutamine proteins, which are retained in the wells. The percent binding is reported for soluble protein alone and for soluble protein plus aggregate from phosphorimager analysis.

To determine whether binding to polyglutamine repeat disease proteins was a property limited to the htt protein or whether binding, and in theory suppression of aggregation, is applicable to other polyQ containing disease proteins, the in vitro binding of H2/H3 and H3/H4 to ataxin-1 was tested. Binding was observed, again with strongest binding to H2/H3, in a polyQ length dependent manner for both normal range (30Qs) and expanded repeat (92Qs) GST-ataxin1.

Also observed was a mild but steady reduction in the formation of aggregates in Cos-1 cells after co-transfection with HD 104Q EGFP and agents bearing H2/H3 spacers. Aggregation was also reduced when the third domain of the therapeutic agent included H3/H4. Both the labeled “target” polypeptide and the therapeutic agent were expressed from one plasmid (pBud C4) under control of strong mammalian promoters CMV and EF-1 alpha. The molar ratio of expressing polypeptides was assumed 1:1. Cells expressing HD104Q EGFP were counted 48 hours after transfection. Therapeutic agents containing H2/H3 and H3/H4 reduced the formation of fluorescent aggregates by 47% and 33%, respectively. The level of suppression remained the same when the promoters were switched.

An even more dramatic inhibition of aggregation was observed when the molar ratio of expressed polypeptides was changed in favor of the therapeutic agents. 104Q EGFP and suppressers were taken in 1:2 ratio with suppressers for co-transfection experiments with two plasmids. Both polypeptides were expressed from pcDNA 3.1 (Invitrogen) under control of CMV promoter. After 48 hours, the number of aggregates formed was reduced by 54%, 74%, and 93% for 104QEGFP co-expressed with H1/H2, H2/H3 and H3/H4 suppressors, respectively. Aggregates that were formed early (within 48 hours after transfection) and released into the medium due to the cell death obviously escaped detection. When cell-free aggregates were collected from the media and counted, we observed a reduction in the number of aggregates by 71%, 90%, and 94% for cells co-expressing HD104Q EGFP with H1/H2, H2/H3 and H3/H4 suppressors, respectively. The reduction in number of early-formed inclusions suggests that the delay in aggregation was caused by the therapeutic agents.

### Example 2

### A Tri-domain Therapeutic Agent Inhibits Pathogenesis in a Drosophila Model of Polyglutamine Repeat Disease

The experiments that follow were designed to test the hypothesis that a symmetrical molecule with two non-pathogenic length polyglutamine tracts (25Qs each), separated by a spacer region, could each interact with cellular proteins containing extended regions of polyglutamine and, further, that if the spacer region contained α-helices angled with respect to each other, the spacer would prevent interaction between the expanded repeat and other polyglutamine repeats. As a result, such a synthetic molecule would suppress polyglutamine repeat-mediated aggregation.

Therapeutic agents were thus designed based on the known crystal structure of the C-terminal region of the TATA box-binding protein (TBP). These agents inhibited aggregation in cell culture when co-expressed with expanded polyglutamine repeats containing truncated huntingtin and bound directly to huntingtin protein in vitro. One of the agents was also expressed in a Drosophila model of polyglutamine repeat pathogenesis and, as described below, found to reduce lethality and neuronal degeneration. In addition, the agent caused a decrease in the number of neurons containing aggregates. These studies demonstrate that the mechanism underlying aggregation has a role in pathogenesis, and they clearly show the potential of therapeutic agents designed to inhibit protein interaction in Huntington's disease, other polyglutamine repeat diseases, and disorders associated with undesirable protein-protein interactions.

Polyglutamine repeat mediated lethality is reduced in vivo by co-expression of a synthetic suppressor: As described above, a therapeutic approach to alleviating pathogenesis in polyQ repeat diseases includes the administration of compounds that bind to the polyQ repeat protein and inhibit further aggregation. To demonstrate that the therapeutic agents described herein are effective in vivo, they were expressed in neurons in a Drosophila model of polyglutamine repeat pathogenesis.

A Drosophila model expressing different polyQ peptides has been described (Marsh et al., Human Mol. Genetics 9:13-25, 2000). In that model, expanded polyQ chains alone were intrinsically cytotoxic and caused neuronal degeneration and early adult. PolyQ peptides, comprised of a glutamine tract+/−a myc/flag C-terminal epitope tag were placed under the control of an upstream activator sequence (UAS). Transgenic flies carrying a polyQ peptide were then crossed to flies expressing the yeast GAL4 transcriptional activator under tissue specific control. For these studies, polyQ peptides were expressed in all neurons from embryogenesis onward by an elav-GAL4 driver.

Drosophila were transformed with the two strongest agents of aggregation, H2/H3 and H3/H4 under UAS control. Several different lines carrying H3/H4 were obtained and expressed in neurons. No effect upon viability was observed when H3/H4 was expressed in neurons (elav-GAL4). It was not possible to obtain transformants with H2/H3, presumably due to toxicity to the organism. Although there is no expression of the transgene until the line is crossed to an appropriate GAL4 line, some read through transcription occurs when constructs are initially injected.

A Q108 line (Q108-16), which shows approximately 20% survival at 25° and 0% survival at 27° was first tested for rescue; GAL4 expression is increased at higher temperatures. Flies expressing the peptide die prior to reaching third instar larvae. As it was desirable to co-express both peptides (Q108 and H3/H4) simultaneously, H3/H4 lines (Su17 and 14 representing different integration events) were crossed into the Q108-16 background prior to expression. When Q108-16 was expressed in neurons, 1% survival of offspring was observed at 27°. When co-expressed with Su17, 53% expected offspring survived and with Su14, 47% survival was observed. At 290, lethality is more severe and 25% and 11% survival was observed, respectively. One suppressor line, Su3B, did not suppress lethality.

More severe Q108 expressing lines were also tested. For these lines, the presence of the agent was not sufficient to rescue polyQ mediated lethality. Therefore, milder lines expressing 48Q (with a C-terminal epitope tag) were tested for rescue of lethality. Two different lines, Q108tag-13 and Q48tag-36, showed significant reduction of lethality by co-expression of H3/H4.

Photoreceptor neuronal cell death is rescued by co-expression of suppressor: When the Q108 peptide, with or without the epitope tag, is expressed by elav-GAL4 in neuronal cells behind the furrow of the eye, an eye with normal external morphology but extensively disrupted internal organization results. Eye sections from flies expressing the 108Q+tag exhibited severe degeneration of the photoreceptor neurons compared to a wild type ommatidia with the regular trapezoidal arrangement of seven visible rhabdomeres. Quantitation of the number of rhabdomeres reveals an average of three remaining per ommatidia in the 108Qtag flies. When co-expressed with H3/H4 in neurons (elav-GAL4), dramatic rescue to near wild-type levels was observed. Lines co-expressing Su14 produce ommatidia with an average of five photoreceptor neurons and the majority of lines co-expressing Su17 produce ommatidia containing the wild-type number of seven photoreceptor. Similar results were found when Su17 was co-expressed with two different 48Qtag lines.

Aggregation in neurons is altered by the presence of suppressor protein: The results described above show that co-expression of an agent designed to inhibit aggregation reduces lethality of Drosophila mediated by an expanded polyglutamine repeat peptide in neurons. In addition, this same suppressor rescues the degeneration of neurons in the eye. Previous studies in Drosophila showing suppression of the polyQ mediated pathogenic phenotype by overexpression of chaperone proteins appeared to produce no overt disruption or reduction in aggregation in eye discs. We therefore investigated whether aggregation in vivo, particularly in neurons, was reduced in a similar manner as in cell culture.

Expression and subcellular localization was first evaluated for lines expressing suppressor alone. When Su17, 14 and 3b was expressed in developing discs (ptc-GAL4), H3/H4 protein stained with myc antibody in all three lines at roughly similar levels. In both wing disc and salivary gland, suppressor protein was localized to the cytosol. Similarly, when H3/H4 was expressed in CNS and ventral ganglia neurons (elav-GAL4), diffuse cytosolic expression in a subset of neurons was observed. In contrast, when the 108Qtag peptide was expressed in discs, as described previously, immunoreactive protein was localized exclusively to the nucleus in salivary gland and formed both nuclear and cytoplasmic aggregates in wing discs. Following expression of 108Qtag in neurons, a distinct distribution pattern was observed. In a subset of neurons, the expanded repeat peptide is diffusely localized to the cytosol, with large, single, primarily perinuclear and cytosolic aggregates. In regions of the central nervous system representing terminally differentiated, mature neurons, tightly formed single nuclear inclusions and diffuse nuclear staining were visible. No cytosolic staining was observed in this neuronal region.

A number of embodiments of the invention have been described. Nevertheless, it will be understood that various modifications may be made without departing from the spirit and scope of the invention. Accordingly, other embodiments are within the scope of the following claims.

WHAT IS CLAIMED IS:

1. A therapeutic agent comprising
(a) a first domain that binds a first protein, the first protein having at least seven consecutive glutamine residues;
(b) a second domain that binds a second protein, the second protein having at least seven consecutive glutamine residues; and
(c) a third domain that separates the first domain from the second domain.

2. The therapeutic agent of claim 1, wherein the first protein and the second protein each have at least seven consecutive glutamine residues.

3. The therapeutic agent of claim 2, wherein the first protein and the second protein each have more than 37 consecutive glutamine residues.

4. The therapeutic agent of claim 1, wherein the first domain and the second domain are identical.

5. The therapeutic agent of claim 1, wherein the first domain or the second domain comprises a peptide.

6. The therapeutic agent of claim 5, wherein the peptide comprises at least three consecutive glutamine residues.

7. The therapeutic agent of claim 6, wherein the peptide comprises the first 17 amino acid residues of a huntingtin protein fused to 25 glutamine residues.

8. The therapeutic agent of claim 1, wherein the third domain comprises a peptide or other polymer.

9. The therapeutic agent of claim 8, wherein the peptide comprises an alpha-helical region or a beta-sheet.

10. The therapeutic agent of claim 6, wherein the third domain comprises LEGLVLTHQQFSSYEPELFPGLIYRMIKPRIVLLIFVSGKVVLTGAKVR-AEIYEAFENIYPILKGFRK (SEQ ID NO: 11).

11. A therapeutic composition comprising the therapeutic agent of claim 1.

12. An isolated DNA molecule, wherein the DNA molecule encodes a polypeptide having three domains:
(a) a first domain that binds a first protein, the first protein having at least seven consecutive glutamine residues;
(b) a second domain that binds a second protein, the second protein having at least seven consecutive glutamine residues; and
(c) a third domain that separates the first domain from the second domain.

13. An expression vector comprising the isolated DNA molecule of claim 12.

14. A cell comprising the expression vector of claim 13.

15. A method of treating a patient who has a disease associated with expanded CAG repeats, the method comprising administering to the patient the therapeutic agent of claim 1.

16. The method of claim 15, wherein the disease is Huntington's disease (HD), primal and bulbar muscular atrophy (SBMA), dentatorubral-pallidoluysian atrophy, spinocerebellar ataxia type 1, type 2, type 6, or type 7, or Machado-Joseph disease (MJD/SCA3).

17. A method of treating a patient who has a disease associated with expanded CAG repeats, the method comprising administering to the patient the DNA molecule of claim 12.
